(19) United States
(12) Patent Application Publication
(10) Pub. No.: US 2026/0198811 A1
(43) Pub. Date: Jul. 16, 2026

(54) RAMAN SIGNAL ANALYSIS APPARATUS USING BEAM SAMPLING AND CONTROL METHOD THEREOF

(71) Applicant: Apollon Inc., Cheongju-si (KR)

(72) Inventors: Aram HONG, Seoul (KR); Miyeon JUE, Seoul (KR); Young Kyu KIM, Seoul (KR)

(21) Appl. No.: 19/450,427

(22) Filed: Jan. 15, 2026

(30) Foreign Application Priority Data
Jan. 15, 2025 (KR) 10-2025-0006122

(51) Int. Cl. A61B 5/1455; A61B 5/00; G01J 3/44

(52) U.S. Cl. CPC A61B 5/1455; A61B 5/7203; G01J 3/44; A61B 2562/0233

(57) ABSTRACT

The present disclosure relates to a Raman signal analysis apparatus using beam sampling. The apparatus includes a beam sampler disposed in a light path irradiated from a light source unit to a target to reflect first light corresponding to a first fraction of light to be used for a sampling purpose, and transmit second light corresponding to a second fraction of the light toward the target. A light distribution unit selectively reflects or transmits third light, which is reflected or scattered from the target by the second light, to output the third light to a plurality of paths. A control module is configured to analyze biometric information associated with the target based on a Raman signal of the light output from the plurality of paths, sample the reflected first light, and correct the Raman signal based on the sampling results.

### CROSS-REFERENCE TO RELATED APPLICATION(S)

This application claims priority from Korean Application No. KR 10-2025-0006122 filed Jan. 15, 2025, which is incorporated herein by reference in its entirety.

### TECHNICAL FIELD

The present disclosure relates to a Raman signal analysis apparatus, and more particularly, to an apparatus for analyzing a Raman signal using a beam sampling technology.

### RELATED ART

A continuous glucose monitoring device is a medical device in which a sensor is attached to a patient (or a general user) for a long period of time to measure the patient's blood glucose during that period, check the increasing and decreasing trends of blood glucose, and provide information to allow the patient to control their diet or determine the timing of injection of medications such as insulin.

For this reason, domestic and foreign diabetes and endocrine societies have revised guidelines to recommend the use of continuous glucose monitoring devices regardless of the type of diabetes for more appropriate health management of diabetic patients.

Recently, devices for analyzing Raman signals in a non-invasive manner have been introduced. However, there is a problem in that measurement values may vary depending on the output and uniformity of light irradiated to a target.

### SUMMARY

An object of the present disclosure is to provide a Raman signal analysis apparatus using beam sampling.

In addition, another object of the present disclosure is to provide a Raman signal analysis apparatus capable of sampling a part of light irradiated from a light source and analyzing biometric information of a target using the sampling result.

The problems to be solved by the present disclosure are not limited to the problems mentioned above, and other problems not mentioned will be clearly understood by those skilled in the art from the following description.

To achieve the above-described objects, a Raman signal analysis apparatus using beam sampling according to an embodiment of the present disclosure may include a light source unit including a single light source that irradiates light to a target; a beam sampler disposed in a light path from the light source unit to the target, the beam sampler being configured to reflect first light corresponding to a first fraction of the light to be used for a sampling purpose and transmit second light corresponding to a second fraction of the light toward the target; a light distribution unit that selectively reflects or transmits third light, which is reflected or scattered from the target by the second light, and outputs the third light to a plurality of paths; and a control module configured to analyze biometric information associated with the target based on a Raman signal of the light output from the plurality of paths, sample the reflected first light, and correct the Raman signal based on the sampling results.

In addition, a control method of a Raman signal analysis apparatus using beam sampling according to an embodiment of the present disclosure for achieving the above-described objects may include irradiating, by a light source unit including a single light source, light to a target; reflecting, by a beam sampler disposed in a light path from the light source unit to the target, first light corresponding to a first fraction of the irradiated light, and transmitting, by the beam sampler, second light corresponding to a second fraction of the irradiated light toward the target; selectively reflecting or transmitting, by a light distribution unit, third light, which is reflected or scattered from the target by the second light, to output the third light to a plurality of paths; sampling the reflected first light; correcting a Raman signal of light output from the plurality of paths based on the sampling results; and analyzing biometric information associated with the target based on the corrected Raman signal and sampling the reflected first light.

In addition, a computer program stored in a non-transitory computer-readable recording medium for executing the method for implementing the present disclosure may be further provided.

In addition, a non-transitory computer-readable recording medium having stored therein a computer program for executing the method for implementing the present disclosure may be further provided.

According to the means for solving the problems described above, the effect of providing a Raman signal analysis apparatus using beam sampling is provided. In addition, according to the means for solving the problems described above, it is possible to sample a part of light irradiated from a light source and analyze biometric information of a target using the sampling result.

The effects of the present disclosure are not limited to the effects mentioned above, and other effects not mentioned will be clearly understood by those skilled in the art from the following description.

### BRIEF DESCRIPTION OF THE DRAWINGS

FIG. 1 is a block diagram of a Raman signal analysis apparatus according to an embodiment of the present disclosure.

FIG. 2 is a plan view of a Raman signal analysis apparatus according to an embodiment of the present disclosure.

FIG. 3 is a flowchart illustrating a control method of a Raman signal analysis apparatus according to an embodiment of the present disclosure.

FIG. 4 is a schematic view illustrating a band-type continuous glucose monitoring device.

### DETAILED DESCRIPTION

Hereinafter, the same reference numerals refer to the same components throughout the present disclosure. The present disclosure does not describe all elements of the embodiments, and descriptions of general contents in the technical field to which the present disclosure pertains or overlapping contents between embodiments are omitted. Terms such as “unit, module, member, and block” as used in the specification may be implemented as software or hardware, and according to embodiments, a plurality of “units, modules, members, and blocks” may be implemented as a single component, or a single “unit, module, member, and block” may include a plurality of components.

Throughout the specification, when a part is referred to as being “connected” to another part, this includes not only a case where it is directly connected but also a case where it is indirectly connected, and the indirect connection includes a connection through a wireless communication network.

In addition, when a part is referred to as “including” a component, this means that it may further include other components rather than excluding other components unless specifically stated to the contrary.

Throughout the specification, when a member is disposed “on” another member, this includes not only a case where a member is in contact with another member but also a case where another member exists between two members.

Terms such as first and second are used to distinguish one component from another component, and the components are not limited by the terms described above.

Singular expressions include plural expressions unless the context clearly indicates otherwise.

In each step, reference numerals are used for convenience of description, and the reference numerals do not describe the order of the steps, and each step may be performed differently from the specified order unless the context clearly states a specific order.

Hereinafter, the operating principles and embodiments of the present disclosure will be described with reference to the accompanying drawings.

In the present specification, the “Raman signal analysis apparatus according to the present disclosure” includes all various devices capable of performing computational processing and providing results to a user. For example, the Raman signal analysis apparatus according to the present disclosure may include all of a computer, a server device, and a portable terminal, or may be in the form of any one thereof.

Here, the computer may include, for example, a notebook, a desktop, a laptop, a tablet PC, a slate PC, or the like equipped with a web browser.

The server device may be a server that processes information by communicating with an external device, and may include an application server, a computing server, a database server, a file server, a game server, a mail server, a proxy server, a web server, and the like.

The portable terminal may be, for example, a wireless communication device that ensures portability and mobility, and may include any kinds of handheld-based wireless communication devices, such as PCS, GSM, Personal Digital Cellular (PDC), Personal Handyphone System (PHS), Personal Digital Assistant (PDA), International Mobile Telecommunication (IMT)-2000, Code Division Multiple Access (CDMA)-2000, W-Code Division Multiple Access (W-CDMA), Wireless Broadband Internet (WiBro) terminal, and smartphone, and wearable devices such as watches, rings, bracelets, anklets, necklaces, glasses, contact lenses, or head-mounted-devices (HMD).

Hereinafter, embodiments of the present disclosure will be described in detail with reference to the accompanying drawings.

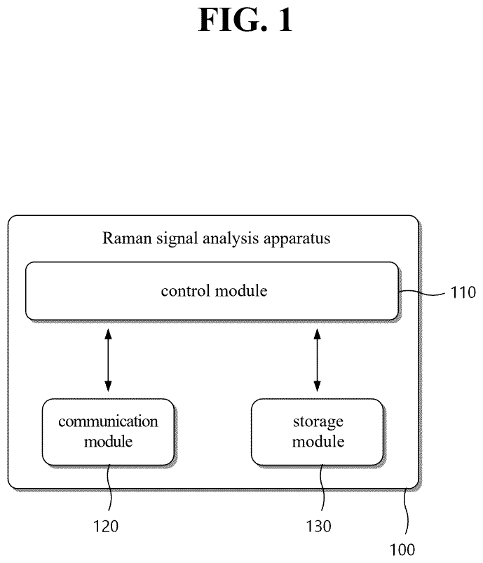

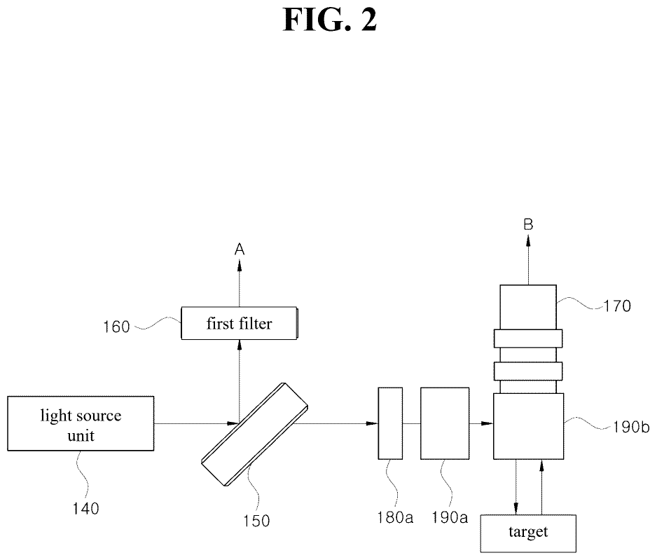

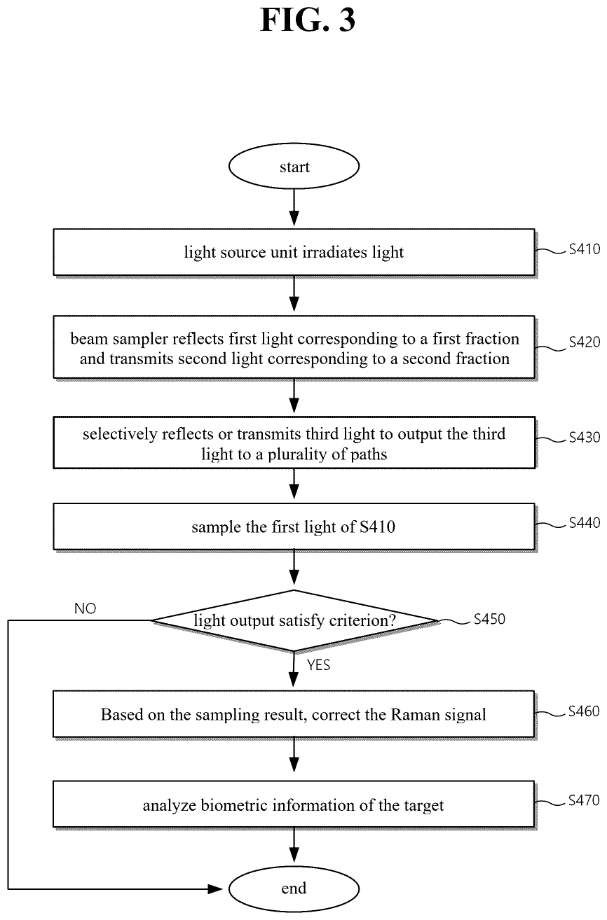

FIG. 1 is a block diagram of a Raman signal analysis apparatus according to an embodiment of the present disclosure. FIG. 2 is a plan view of a Raman signal analysis apparatus according to an embodiment of the present disclosure. FIG. 3 is a flowchart illustrating a control method of a Raman signal analysis apparatus according to an embodiment of the present disclosure.

Referring to FIG. 1, a Raman signal analysis apparatus 100 according to an embodiment of the present disclosure may include a control module 110, a communication module 120, and a storage module 130. These components are related to the operation control of the Raman signal analysis apparatus 100, and other components will be described with reference to FIG. 2. However, in some embodiments, the Raman signal analysis apparatus may include fewer or more components than those shown in FIG. 1.

Referring to FIG. 2, the Raman signal analysis apparatus 100 according to an embodiment of the present disclosure may include a light source unit 140, a beam sampler 150, a first filter 160, a light distribution unit 170, a lens 180, and one or more mirrors 190 (190a and 190b). However, in some embodiments, the Raman signal analysis apparatus may include fewer or more components than those shown in FIG. 2.

Hereinafter, each component and operation process of the Raman signal analysis apparatus 100 according to an embodiment of the present disclosure will be described in detail with reference to FIGS. 1 and 2. The components of the Raman signal analysis apparatus shown in FIGS. 1 and 2 may be disposed inside a main body or a housing that forms an internal accommodation space, but the embodiment is not limited thereto.

The light source unit 140 may be disposed within the housing and may include a single light source for irradiating light to a target. In an embodiment of the present disclosure, the target may refer to a subject or a patient. The wavelength emitted and irradiated from the light source unit 140 may vary depending on the purpose of measurement. For example, the light source unit 140 may be a laser diode.

When the Raman signal analysis apparatus 100 according to the present disclosure is mounted so that the contact surface is in contact with the body of the target (subject), the light emitted to the outside may reach the subject (e.g., skin). The light that reaches the subject may be reflected or scattered and may then return.

The control module 110 may be configured to control the light source unit 140 to irradiate light (S410).

A beam sampler 150 may be provided in a path where the light irradiated from the light source unit 140 proceeds. The beam sampler 150 may be provided in the paths of the light irradiated from the light source unit 140 to the target and may reflect at least a portion of the light irradiated from the light source unit 140 in order to perform a sampling process.

The beam sampler 150 may reflect first light corresponding to a first fraction of the light irradiated from the light source unit 140 and may transmit second light corresponding to a second fraction (S420). More specifically, the beam sampler 150 may reflect the first light corresponding to the first fraction of the irradiated light and may transmit the second light corresponding to the second fraction of the irradiated light toward the target.

The first light reflected from the beam sampler 150 may be directed to path A and input to a photodetector element, and the control module 110 may proceed with sampling using the input first light.

In some embodiments, the apparatus 100 may further include a first filter 160 that filters the light reflected from the beam sampler 150 so that the first light is input to the photodetector element. For example, the first light corresponding to the first fraction irradiated from the light source unit 140 and reflected by the beam sampler 150 may be input to the photodetector element. For example, the light corresponding to the first fraction irradiated from the light source unit 140 and reflected by the beam sampler 150 may pass through the first filter 160, and the first light may be input to the photodetector element. By way of example, the first filter 160 may be implemented as an ND filter.

A lens 180a and a first mirror 190a may be provided between the beam sampler 150 and where the light is to be irradiated to the target, and a second mirror 190b for reflecting light to direct the light to the skin of the target may be provided in the corresponding area. In other words, the light transmitted through the beam sampler 150 may be condensed through the lens 180a; the light condensed through the lens 180a may pass through the mirror 190a, may be reflected by the mirror 190b, and may reach the target.

Subsequently, the light reflected or scattered from the target after reaching the target may be directed to the light distribution unit 170. The light distribution unit 170 may selectively reflect or transmit third light reflected or scattered from the target by the second light and may output it to at least one path. The light distribution unit 170 may selectively reflect or transmit the third light to output the third light to a plurality of paths (S430). FIG. 2 depicts path B by the light distribution unit 170.

Although path B is illustrated as a path by the light distribution unit 170 in FIG. 2, it is not limited to one path, and a plurality of paths may be included. For example, the at least one path may include path B, path C, or path D. Each path may be designated for measuring different indicators. When the plurality of paths include path B, path C, and path D, the light distribution unit 170 may include at least one beam splitter, a second filter, a third filter, and a lens. For example, to configure a further path, a beam splitter and a lens may be additionally included after path C.

The third light may be directed to a first beam splitter, and the first beam splitter may reflect at least a portion of the third light to be directed to path B and may transmit at least a portion of the light. The light moving to path B may be input to a photodetector element, and the control module 110 may be configured to analyze it.

A lens and a third filter may be provided in path B. The third light reflected by the first beam splitter and directed to path B may pass through the third filter and the lens and may be directed to the photodetector element. The third light transmitted through the first beam splitter may be directed to a mirror.

In some embodiments, at least one filter may be provided between the mirror 190b and the first beam splitter. For example, a second filter may be provided between the mirror 190b and the second beam splitter. The second filter may block light (e.g., laser beam) reflected and incoming from the skin of the target. The second filter may be distinguished from the wavelength band of the Raman signal and the wavelength band of the reflected light, and may block light (e.g., laser beam) reflected and incoming from the skin of the target.

Subsequently, the third light may be directed to a mirror, and the third light reflected by the mirror may be directed to path C. When configured to include an additional path after path C, a beam splitter may be provided instead of the mirror 190b. The light moving to path C may be input to a photodetector element, and the control module 110 may be configured to analyze it. A lens and a third filter may be provided in path C.

The control module 110 may be configured to sample the first light (S440). The control module 110 may be configured to check whether the light output in the sampling result of S400 meets a criterion (S450). Based on the sampling result of S440, the control module 110 may be configured to correct the Raman signal (S460). Based on the corrected Raman signal, the control module 110 may be configured to analyze the biometric information of the target (S470).

The control module 110 may be configured to analyze the biometric information of the target (subject) based on the Raman signal obtained from the photodetector element. The control module 110 may be configured to analyze the biometric information of the target based on the Raman signal obtained for the target. The control module 110 may be configured to extract the biometric information of the target through a peak area value of a Raman spectrum range of at least one of glucose, protein, ketone, alcohol, caffeine, lactic acid, or fat associated with the target.

The control module 110 may be configured to receive light reflected or scattered from the target, obtain a Raman signal for Raman signal analysis, and generate a Raman spectrum. For such operation, the Raman signal analysis apparatus 100 may include at least one component among a lens for concentrating light reflected or scattered from the target in one place, an optical filter for filtering a partial wavelength band of light, a mirror for changing the proceeding direction of light, or a spectrometer for dispersing light by wavelength band to generate a spectrum of light.

The Raman signal analysis apparatus 100 may include at least one component that changes at least one of the proceeding direction, wavelength, polarization, and light quantity of light, or disperses light by wavelength band to generate a spectrum in order to receive light reflected or scattered from the subject and generate a Raman spectrum.

The control module 110 may be configured to calculate the output of the light irradiated from the light source based on the result of sampling the first light. In some embodiments, the control module 110 may be configured to calculate (e.g., determine, evaluate, detect, or measure) the output of the light irradiated from the light source based on the result of sampling the first light, and in this case, the calculated output of light may include the first light, the second light, and light absorbed by the beam sampler 150.

The control module 110 may be configured to control the measurement to stop if the calculated output of light does not satisfy a preset criterion, and in response thereto, may output/transmit an error message.

In some embodiments, the control module 110 may be configured to correct the Raman signal according to the second light corresponding to the second fraction based on the calculated output of light.

In an embodiment of the present disclosure, the storage module 130 may store analysis criteria for analyzing the biometric information of the subject based on the Raman signal. In some embodiments, the control module 110 may be configured to reflect a correction value in the analysis criteria based on the measured output of the light source unit 140.

For example, the analysis criteria may include a mathematical equation. For example, the control module 110 may be configured to adjust variables of the mathematical equation based on the sampling result.

In some embodiments, the control module 110 may be configured to generate a biometric information analysis result associated with the target (subject) based on the Raman signal, and the control module 110 may be configured to correct the analysis result based on the sampling result.

The control module 110 may be configured to measure and/or calculate uniformity for the light output of the light source based on the result of sampling the first light. The control module 110 may be configured to check whether the measured and/or calculated uniformity satisfies a predetermined uniformity requirement. If the measured and/or calculated uniformity does not satisfy the predetermined uniformity, the control module 110 may be configured to control the measurement to stop and output or transmit an error message.

In some embodiments, the control module 110 may be configured to calculate a probability that an error exists in the biometric information analysis result based on the result of sampling the first light.

The storage module 130 may store criteria for accurately measuring biometric information analysis, and such criteria may apply a range of light output values of the light source and uniformity for the light output.

In other words, the control module 110 may be configured to evaluate that there is almost no probability that an error exists when the light output value of the light source unit 140 and the uniformity for the light output accurately meet the criteria as a result of sampling the first light.

The control module 110 may obtain a 1125 cm⁻¹ Raman signal by installing a 915 nm wavelength band filter in front of a photodetector such as an avalanche photodiode (APD), photodiode (PD), charge-coupled device (CCD), or complementary metal-oxide-semiconductor (CMOS) to measure the 1125 cm⁻¹ peak, which is a Raman signal peak for measuring blood glucose from an 830 nm light source.

If a light source having a different wavelength band is used, the control module 110 may obtain a Raman signal of 1815 cm⁻¹, if a light source of 785 nm is used, and may obtain a Raman signal of 840 cm⁻¹, if 850 nm is used. As Raman signals of more regions are obtained, information on the in vivo concentration of a specific biological substance can be more accurately calculated.

In some embodiments, the control module 110 may be configured to apply light sources and optical filters of various wavelength bands to obtain Raman signals of each biometric information (e.g., glucose, protein, ketone, alcohol, caffeine, lactic acid, and fat).

The light source used in the embodiment of the present disclosure may be red light of 600 nm or more and/or near-infrared rays having high light permeability in a living body for ease of measurement of substances in a living body. After the wavelength band having the greatest influence is set as a combination of a single light source and a single filter, a wavelength longer and shorter than the basic light source wavelength by a preset range (e.g., 10 nm) may be additionally applied. In addition, a light source wavelength for use in background signal removal and calibration may be selected by measuring the Raman signal wavelength band of another biological substance (e.g., 1450 cm⁻¹ for protein).

The Raman signal analysis apparatus 100 according to the present disclosure may include a battery for driving the above-described components. The battery may be detachable and may be disposed outside the housing, but the present disclosure is not limited thereto.

The control module 110 may be configured to enable generation of a Raman spectrum through an electrical signal. For example, each photodetector element of the photodetector element array may be implemented as a Charge Coupled Device (CCD), an avalanche photodiode (APD) array, a photodiode (PD), and/or a complementary metal-oxide-semiconductor (CMOS), etc., but the present disclosure is not limited thereto.

The control module 110 may be configured to generate a Raman spectrum based on the signal generated by the photodetector element array. The Raman spectrum may be generated in the form of a graph in which the x-axis represents a Raman shift value (unit: cm⁻¹) and the y-axis represents signal intensity.

The control module 110 may be configured to analyze the generated Raman spectrum to measure the biometric information of the subject, and may be configured to extract the biometric information of the subject through a peak area value of a Raman spectrum range of at least one of glucose, protein, ketone, alcohol, caffeine, lactic acid, fat, or any combinations thereof for the subject.

For example, taking blood glucose as an example, the control module 110 may be configured to perform a calibration process on the Raman spectrum specific to blood glucose and skin constituent proteins before measuring the blood glucose of the subject.

In some embodiments, during the calibration, the control module 110 may be configured to reduce noise in the generated spectrum through Savitzky-Golay filtering and may remove the background of the generated spectrum through polynomial fitting. A polynomial fitting order suitable for background removal may be determined based on the intensities of four wavelengths, namely, the initial wavelength, the wavelength at the 2/4 point, the wavelength at the ¾ point, and the end wavelength.

When starting the operation of the device, restarting after stopping blood glucose measurement, or re-wearing the device after putting it off, the control module 110 may be configured to perform calibration again.

The user may check an error message through an external terminal connected to the Raman signal analysis apparatus 100 according to the present disclosure. The error message may include text or an image requesting a change of attachment site or re-attachment.

Meanwhile, if the intensity ratio between general Raman signal peaks and the intensity ratio between acquired Raman signal peaks differs by more than a certain standard, the control module 110 may be configured to determine that there is poor contact between the subject and the device, and may cause the communication module 120 to transmit an error message to the external terminal.

However, the present disclosure is not limited thereto, and the control module 110 may be configured to display the error message through the output unit 140 included in the continuous glucose monitoring device without transmitting the above-described error message to the external terminal.

Further, the control module 110 may be configured to utilize machine learning techniques such as partial least squares (PLS), Support vector machine (SVM), or deep learning utilizing Autoencoder, ResNet, and the like, to learn the area of each peak of glucose, protein, fat, ketone, alcohol, lactic acid, caffeine, etc. and the glucose level at the time of measurement as data, and may be configured to continuously measure various biometric information including the blood glucose of the subject based on the learned model.

In an embodiment, the glucose level may be measured through methods such as finger blood sampling, venous blood sampling, continuous CGM, and the like, but the glucose level measurement method is not limited thereto.

In some embodiments, the control module 110 may be configured to estimate the amount of glucose in the interstitial fluid based on the ratio of the area of the peak centered at 1450 cm⁻¹ and the area of the peak centered at 1660 cm⁻¹ to the area of the peak centered at 1125 cm⁻¹. Unless specifically mentioned otherwise, the Raman peaks disclosed hereinbelow may be understood as the amount of Raman shift in response to using the 830 nm light source.

Here, in the case of the peak centered at about 1450 cm⁻¹, which is a peak corresponding to protein, the area may be obtained using the range of 1415 cm⁻¹ to 1480 cm⁻¹.

Meanwhile, in the case of the peak centered at about 1660 cm⁻¹, which is a peak corresponding to fat, the area may be obtained using the range of 1630 cm⁻¹ to 1685 cm⁻¹.

Meanwhile, in the case of the peak centered at about 1125 cm⁻¹, which is a peak corresponding to glucose, the area may be obtained using the range of 1100 cm⁻¹ to 1145 cm⁻¹.

In an embodiment, in the case of the peak centered at about 1725 cm⁻¹, which corresponds to ketone, the control module 110 may be configured to obtain the area corresponding to ketone using the range of 1700 cm⁻¹ to 1750 cm⁻¹.

In an embodiment, in the case of the peak centered at about 1200 cm⁻¹, which corresponds to alcohol, the control module 110 may be configured to obtain the area corresponding to alcohol using the range of 1180 cm⁻¹ to 1220 cm⁻¹.

In an embodiment, in the case of the peak centered at about the 1650 cm⁻¹, which corresponds to carbon-oxygen double bond of caffeine (carbonyl group of caffeine molecule), the control module 110 may be configured to obtain the area using the range of 1600 cm⁻¹ to 1700 cm⁻¹.

In an embodiment, in the case of the peak centered at about 1725 cm⁻¹, which corresponds to carboxyl group for lactic acid, the control module 110 may be configured to obtain the area using the range of 1700 cm⁻¹ to 1750 cm⁻¹.

In some embodiments, if a 915 nm wavelength band filter is installed in front of a photodetector such as APD, CMOS, CCD, or APD array to measure the 1125 cm⁻¹ peak, which is a Raman signal peak for measuring blood glucose in response to an 830 nm light source, the control module 110 may be configured to obtain a 1125 cm⁻¹ Raman signal. Thereafter, if a 930 nm filter is used in another photodetector, a Raman signal of 1295 cm⁻¹ may be obtained from the same light source, and if a 900 nm filter is used, a Raman signal of 937 cm⁻¹ may be obtained using the same 830 nm light source. As Raman signals of more regions are acquired, information on the in vivo concentration of a specific biological substance can be more accurately calculated.

At this time, the combination of filters and light sources used may be set such that the combination of a single light source and a single filter for the wavelength band where the Raman signal of a specific biological substance has the greatest influence is set as the basic filter wavelength, and then one or more wavelengths that are longer and shorter than the basic filter wavelength may be used. In order to obtain more information on the wavelength band where the influence of a specific biological substance is greatest, a filter in a range within 10 nm from the basic filter wavelength may be used, and a filter of a wavelength for use in background signal removal and calibration may be selected by measuring the Raman signal wavelength band of other biological substances (e.g., protein, 1450 cm⁻¹).

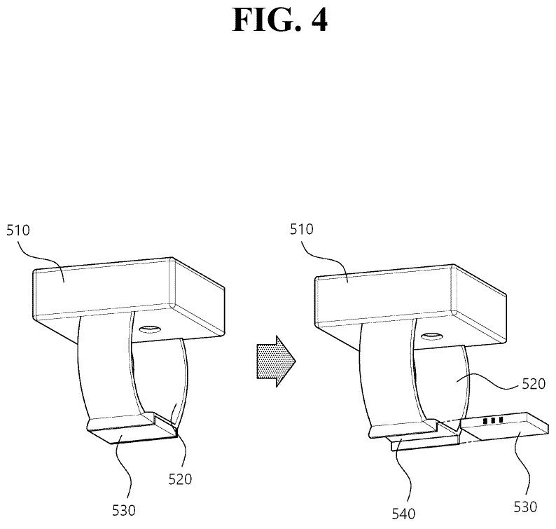

FIG. 4 is a schematic view illustrating a band-type continuous glucose monitoring device. The continuous glucose monitoring device including or applying the Raman signal analysis apparatus 100 according to the present disclosure may be implemented in a band form that can be fixed to a wrist, ankle, forearm, or the like. To this end, the continuous glucose monitoring device may include a housing 510 and a band 520.

Furthermore, the continuous glucose monitoring device according to the present disclosure may include a battery 530. The battery 530 may be disposed separately from the housing 510. For example, the battery 530 may be disposed opposite from the housing 510.

Meanwhile, a circuit that electrically connects the battery 530 and the components in the housing 510 may be disposed in the band 520.

The battery 530 may be formed to be detachable from the band 520, and for this purpose, the band may include a battery fastening part 550. The battery fastening part 550 may fix the battery 530 to the band and electrically connect the battery 530 and the circuit disposed in the band 520.

Meanwhile, an auxiliary battery may be disposed in the housing 510. The auxiliary battery may allow the continuous glucose monitoring device to maintain its function without switching to an off state during replacement of the battery 530. If the device switches to an off state when replacing the battery, calibration must be performed when starting blood glucose measurement after battery replacement. Accordingly, a gap may occur in the blood glucose measurement time, and the user may experience the inconvenience of having to perform calibration every time the battery is replaced. The present disclosure provides a means to improve user convenience by preventing the device from switching to an off state even when replacing the battery.

Herein, the control module may include at least one processor.

The processor may be implemented as a storage unit, which stores an algorithm for controlling the operation of components in the apparatus or data with respect to a program executing the algorithm, and at least one processor, which performs the above-described operation using the data stored in the storage unit. In this case, the storage unit and the processor may be implemented as separate chips. Alternatively, the storage unit and the processor may be implemented as a single chip.

In addition, the processor may control any one or a combination of the components discussed above to implement various embodiments according to the present disclosure described in the drawings below on the present apparatus.

The processor may generally control the overall operation of the apparatus according to the present disclosure in addition to operations related to the application program. The processor may process signals, data, information, or the like that are input or output through the components discussed above or drive an application program stored in the storage unit to provide or process appropriate information or functions to the user.

In addition, the processor may control at least some of the components of the apparatus according to the present disclosure to drive the application program stored in the storage unit. Furthermore, the processor may operate at least two or more of the components included in the apparatus according to the present disclosure in combination with one another for the driving of the application program.

The processor may be implemented as one or more processors. Hereinafter, even if the processor is mentioned in the singular term, it may be considered as plural. The processor can control the components of the Raman signal analysis apparatus. The processor may refer to a data processing device built into hardware having a physically structured circuit to perform functions expressed by codes or instructions included in a program. As such, the processor may cover processing devices such as a microprocessor, a central processing unit (CPU), a processor core, a multiprocessor, an application-specific integrated circuit (ASIC), and a field programmable gate array (FPGA) as examples of data processing devices built into hardware, but the scope of the present disclosure is not limited thereto. The processor may be provided with a separate learning processor for performing artificial intelligence operations or may be provided with a learning processor itself.

In various embodiments, the processor may include one or more of a Central Processing Unit (CPU), an Application Processor (AP), or a Communication Processor (CP). At least a part of the processor may be hardware, such that it may access a memory and perform functions related to instructions stored in the memory.

The communication module may include one or more modules that connect the Raman signal analysis apparatus to one or more networks.

The communication module may include one or more components enabling communication with an external device, and may include, for example, at least one of a broadcast reception module, a wired communication module, a wireless communication module, a short-range communication module, or a location information module.

The wired communication module may include various wired communication modules such as a Local Area Network (LAN) module, a Wide Area Network (WAN) module, or a Value Added Network (VAN) module, as well as various cable communication modules such as Universal Serial Bus (USB), High Definition Multimedia Interface (HDMI), Digital Visual Interface (DVI), recommended standard (RS) 232, power line communication, or plain old telephone service (POTS).

The wireless communication module may include a wireless communication module supporting various wireless communication methods such as Global System for Mobile (GSM) Communication, Code Division Multiple Access (CDMA), Wideband Code Division Multiple Access (WCDMA), Universal Mobile Telecommunications System (UMTS), Time Division Multiple Access (TDMA), Long Term Evolution (LTE), 4G, 5G, 6G, etc., in addition to a Wi-Fi module and a Wireless Broadband (WiBro) module.

The wireless communication module may include a wireless communication interface including an antenna and a transmitter for transmitting communication signals. In addition, the wireless communication module may further include a signal conversion module that modulates a digital control signal output from the processor through the wireless communication interface into an analog wireless signal under the control of the processor.

The short-range communication module is for short-range communication and may support short-range communication using at least one of Bluetooth®, Radio Frequency Identification (RFID), Infrared Data Association (IrDA), Ultra-Wideband (UWB), ZigBee, Near Field Communication (NFC), Wireless-Fidelity (Wi-Fi), Wi-Fi Direct, and Wireless Universal Serial Bus (USB) technologies.

The communication module may use the name of a communication interface. The communication interface can establish communication between the electronic device and an external device. For example, the communication interface can communicate with the external device through wireless communication (e.g., Wi-Fi, Bluetooth, NFC, magnetic stripe transmission (MST), and the like) or wired communication.

The storage module is a means for storing data and may include a configuration such as a database or memory.

The storage module may store data supporting various functions of the apparatus according to the present disclosure. The storage module may store a plurality of application programs (or applications) driven in the apparatus, data for the operation of the apparatus, and instructions. At least some of these application programs may exist for the basic functions of the apparatus. Meanwhile, the application program may be stored in the storage module, installed in the apparatus, and driven to perform an operation (or function) by the processor.

The storage module may store data supporting various functions of the apparatus according to the present disclosure and programs for the operation of the processor, may store input/output data (e.g., music files, still images, videos, etc.), and may store a plurality of application programs driven in the apparatus, data for the operation of the apparatus, and instructions. At least some of these application programs may be downloaded from an external server through wireless communication.

Such a storage module may include at least one type of storage medium among a flash memory type, a hard disk type, a Solid State Disk (SSD) type, a Silicon Disk Drive (SDD) type, a multimedia card micro type, a card type memory (e.g., SD or XD storage module, etc.), a random access memory (RAM), a static random access memory (SRAM), a read-only memory (ROM), an electrically erasable programmable read-only memory (EEPROM), a programmable read-only memory (PROM), a magnetic memory, a magnetic disk, and an optical disk. In addition, the storage module may be separately provided from the apparatus and, for example, may be a database connected by wire or wirelessly.

The memory may be operationally coupled to the processor and may store at least one code executed by the processor. Memory may collectively refer to various types of storage devices. The memory may store information necessary for performing calculations using artificial intelligence, machine learning, and artificial neural networks.

The memory may store various learning models. The learning models stored in the memory may infer a result for new input data other than learning data, and the inferred result may be used as a basis for determination to perform a certain operation. The learning models stored in the memory may be learned based on label information, and various backpropagation algorithms may be applied so that the loss function has a target value in order to increase the accuracy of learning.

The method according to an embodiment of the present disclosure described above may be implemented as a program (or application) to be executed in combination with a server, which is hardware, and stored in a medium.

The above-described program may include codes coded in a computer language such as C, C++, JAVA, machine language, etc. that can be read by a processor (CPU) of the computer through a device interface of the computer so that the computer reads the program and executes the methods implemented as the program. Such codes may include functional codes related to functions defining necessary functions for executing the methods, and may include control codes related to execution procedures necessary for the processor of the computer to execute the functions according to a predetermined procedure. In addition, such codes may further include memory reference-related codes regarding at which location (address) of the internal or external memory of the computer additional information or media necessary for the processor of the computer to execute the functions should be referenced. In addition, when the processor of the computer requires communication with any other remote computer or server to execute the functions, the codes may further include communication-related codes regarding how to communicate with any other remote computer or server using the communication module of the computer and what information or media should be transmitted and received during communication.

The medium for storing refers to a non-transitory medium that stores data semi-permanently and is readable by a device, rather than a medium that stores data for a short moment such as a register, cache, or memory. Specifically, examples of the non-transitory medium for storing include ROM, RAM, CD-ROM, magnetic tape, floppy disk, optical data storage device, etc., but are not limited thereto. That is, the program may be stored in various recording media on various servers accessible by the computer or various recording media on the user's computer. In addition, the medium may be distributed in computer systems connected by a network, so that codes readable by a computer in a distributed manner may be stored.

The steps of the method or algorithm described in connection with the embodiments of the present disclosure may be implemented directly in hardware, in a software module executed by hardware, or by a combination thereof. The software module may reside in a Random Access Memory (RAM), a Read Only Memory (ROM), an Erasable Programmable ROM (EPROM), an Electrically Erasable Programmable ROM (EEPROM), a Flash Memory, a hard disk, a removable disk, a CD-ROM, or any form of computer-readable recording medium well known in the art to which the present disclosure pertains.

Although the embodiments of the present disclosure have been described above with reference to the accompanying drawings, those of ordinary skill in the art to which the present disclosure pertains will understand that the present disclosure can be implemented in other specific forms without changing the technical spirit or essential features thereof. Therefore, the embodiments described above should be understood as illustrative in all respects and not restrictive.

What is claimed is:

1. A Raman signal analysis apparatus using beam sampling, comprising:
a light source unit including a single light source that irradiates light to a target;
a beam sampler disposed in a light path from the light source unit to the target, wherein the beam sampler reflects first light corresponding to a first fraction of the light to be used for a sampling purpose and transmits second light corresponding to a second fraction of the light toward the target;
a light distribution unit that selectively reflects or transmits third light, which is reflected or scattered from the target by the second light, and outputs the third light to a plurality of paths; and
a control module configured to:
analyze biometric information associated with the target based on a Raman signal of the light output from the plurality of paths;
sample the reflected first light; and
correct the Raman signal based on results of said sampling,
wherein the control module is further configured to:
determine an output of the light irradiated from the light source based on the results of sampling the first light; and
correct the Raman signal based on the determined output of the light.

2. The apparatus of claim 1, wherein the control module is configured to correct the Raman signal according to the second light corresponding to the second fraction based on the determined output of the light.

3. The apparatus of claim 1, further comprising:
a storage module that stores analysis criteria for analyzing the biometric information of the target based on the Raman signal,
wherein the control module is configured to reflect a correction value in the analysis criteria based on the determined output of the light source.

4. The apparatus of claim 1, further comprising a first filter that filters light reflected from the beam sampler to allow the first light to be input to a photodetector element.

5. The apparatus of claim 1, wherein the control module is configured to sample the first light to measure uniformity of light output of the light source unit, and check whether the measured uniformity satisfies a predetermined uniformity.

6. The apparatus of claim 1, wherein the control module is configured to sample the first light to determine a probability that an error exists in a result of the biometric information analysis.

7. The apparatus of claim 1, wherein the light distribution unit comprises:
at least one beam splitter that disperses the third light;
at least one second filter that blocks light reflected from the target; and
at least one third filter that separates light of a wavelength to be obtained in at least one path among the plurality of paths.

8. A method of Raman signal analysis using beam sampling, comprising:
irradiating, by a light source unit including a single light source, light to a target;
reflecting, by a beam sampler disposed in a light path from the light source unit to the target, first light corresponding to a first fraction of the light, and transmitting, by the beam sampler, second light corresponding to a second fraction of the light toward the target;
selectively reflecting or transmitting, by a light distribution unit, third light, which is reflected or scattered from the target by the second light, to output the third light to a plurality of paths;
sampling the reflected first light;
correcting a Raman signal of light output from the plurality of paths based on results of said sampling; and
analyzing biometric information associated with the target based on the corrected Raman signal,
wherein an output of the light irradiated from the light source unit is determined based on the results of sampling the first light, and the Raman signal is corrected based on the determined output of the light.

9. A non-transitory computer-readable recording medium having stored therein a computer program for performing the method of claim 8.
